(19) United States
(12) Patent Application Publication
(10) Pub. No.: US 2004/0249243 A1
(43) Pub. Date: Dec. 9, 2004

(54) Tamponade apparatus and method of using same

(76) Inventor: Daniel Eduard Kleiner, New Castle, New South Wales (AU)

Correspondence Address: PEARNE & GORDON LLP, 1801 EAST 9TH STREET, SUITE 1200, CLEVELAND, OH, 44114-3108, US

(21) Appl. No.: 10/489,650

(22) PCT Filed: Sep. 23, 2002
(86) PCT No.: PCT/AU02/01302

(30) Foreign Application Priority Data
Sep. 21, 2001 (AU) PR 7850

(51) Int. Cl. A61B 1/00

(52) U.S. Cl. 600/115

(57) ABSTRACT

Elongate flexible sheath (2) for being driven into position along a body cavity of a patient by an instrument (4) such as an endoscope. Sheath has a closed front end region (10) blocking passagc in longitudinal direction and an opposite rear end region. In form of inflatable balloon, at least a section (18) of front end region transparent to enable viewing into body cavity for endoscope or other driving examination instrument. Sheath provided with one or more conduits (20, 22) lying along balloon and opening to exterior of balloon at front end region enabling fluid delivery, suction, or access of an implement into body cavity. Methods of positioning sheath in body cavity.

### FIELD OF THE INVENTION

The present invention relates to sheaths for use in facilitating examination or treatment within a body cavity of a patient as well as methods of positioning such sheaths along within the body cavity. Various embodiments are provided which find application in medical and veterinary fields.

### BACKGROUND OF THE INVENTION

Tamponade devices incorporating balloons for applying pressure to bleed sites in for instance the gastrointestinal tract upon inflation of the balloon are known in the art. Such devices generally fall into two broad categories, one of which comprises tamponade devices incorporating a balloon carried on a catheter or the like wherein the catheter extends through the balloon for inflation of the balloon laterally and the balloon is removed from the patient with withdrawal of the device from the body of the patient. An example of this type of device is shown in U.S. Pat. No. 4,464,175.

Another category comprises tamponade devices in which the balloon is arranged for being everted from the catheter or like instrument into an extended condition prior to being once inflated laterally for applying pressure to the surrounding wall defining the relevant body cavity and the catheter has been located in position in the body cavity.

An example of a catheter incorporating an everted balloon arrangement is disclosed in patent application EP 0227583 for dilating occluded blood vessels. In particular, the balloon is arranged within a housing for being expelled therefrom by a telescopic sheath slidable longitudinally along the catheter to force the balloon from the catheter in conjunction with the application of fluid pressure to the interior of the balloon. The balloon is everted as it is forced from the catheter and is guided along the interior of the blood vessel by the sheath. When in position, the balloon is inflated to thereby dilate the occlusion.

Further catheters of this general type are disclosed in patent applications WO 96/22122 and DT 2406823, and in patent U.S. Pat. No. 5,328,469.

In U.S. Pat. No. 4,271,839 another catheter for use in dilating occluded blood vessels is disclosed. In this device, an elastic balloon is arranged in an inverted condition in a distal end of the catheter. In use, the catheter is located in close proximity to the occlusion and the balloon everted from the catheter by fluid pressure applied to the interior of the balloon. The balloon is extruded from the catheter in anisotropic fashion in advance of substantial lateral expansion. Upon the balloon being fully extended, the balloon is inflated and thereby expanded laterally into contact with the occlusion to thereby line the surrounding blood vessel wall for effecting dilation of the occlusion. To retract the balloon the fluid pressure within the balloon is lowered and a cord attached to the interior of the free end of the balloon is then retracted causing the balloon to be withdrawn into the catheter.

In International Patent Application No. WO 87/05523 there is disclosed a tubular device incorporating an flexible element arranged in an invaginated conformation for being located along a body orifice or duct. The device incorporates an outer tubular support housing the flexible element and a stiff push tube slidable within the outer support for causing the element to be progressively everted as it is forced from the outer support by the push tube. The push tube, therefore, not only drives the everting of the element but also guides the element along the relevant duct or body orifice as the flexible element is being extended from the outer support.

A further such device described in WO 87/05523 for sealing the nasal fossae comprises a sealed tube in which a balloon is housed in one end thereof and a plunger is received in an opposite end. Upon progression of the plunger along the tube the balloon is caused to be everted and fully extended from the tube. With further progression of the plunger, the balloon is inflated radially to thereby be brought into contact with the surrounding wall defining the body cavity or duct and so fill the surrounding volume. Such devices as disclosed in WO 87/05523 are, therefore, only suitable for following substantially straight paths of progression within the body or in the later type of device, for filling a surrounding void where a bleed site is known to exist.

However, as indicated above, in many situations the site of a bleed within, for instance, the lower gastrointestinal tract is unknown and indeed, the path to the bleed site may be sinuous or deviate significantly from a straight path. Accordingly, it is desirable that methods and apparatus be provided which may be used in such applications.

### SUMMARY OF THE INVENTION

It is an aim of the present invention to ameliorate one or more problems of the prior art or to at least provide a useful alternative.

In one aspect of the present invention there is provided a method of locating an elongated inflatable sheath in position along a body cavity of a patient, wherein the sheath has a closed front end region blocking passage through the sheath in a longitudinal direction thereof and an opposite rear end region, and the method comprises:

(a) introducing an instrument into the body cavity for driving the sheath along the body cavity in an extended condition in which the front end region of the sheath is forward of the rear end region;

(b) feeding the instrument a desired distance into the body cavity whereby the sheath is driven along the body cavity in the extended condition with the rear end region of the sheath drawn along behind the front end region with travel of the instrument along the body cavity; and

(c) withdrawing the instrument from the body cavity leaving the sheath behind in position in the body cavity.

Typically, the sheath will be fitted on the instrument or otherwise be coupled with the instrument prior to the instrument being introduced into the body cavity.

The instrument may be inserted into the sheath for driving the sheath along the body cavity. Preferably, the sheath is an inflatable balloon and the instrument may be inserted directly into an interior lumen of the balloon for being filled with a fluid such as air for causing inflation of the sheath. In this embodiment, the rear end region of the sheath may be adapted for being clamped to the instrument or closed in some other manner such that the lumen is thereby substantially sealed from the atmosphere to enable inflation of the sheath. In another form, the sheath may be provided with an interior passageway that extends from the rear end region of the sheath toward the front end region of the sheath for reception of the instrument and which is separate from the lumen. Alternatively, a sheath may be provided wherein the front end region of the sheath is adapted for being linked with the instrument such that the sheath lies alongside the instrument as the instrument is fed along the body cavity.

Accordingly, in another aspect of the present invention there is provided a method of locating an elongated sheath in position along a body cavity of the patient, wherein the sheath is in the form of an inflatable balloon having a closed front end region blocking passage through the sheath and an opposite rear end region, the method comprising:

(a) fitting the sheath on an instrument for driving the sheath along the body cavity in an extended condition in which the front end region of the sheath is forward of the rear end region, and such that the instrument is received within the sheath;

(b) introducing the instrument into the body cavity; and

(c) feeding the instrument a desired distance into the body cavity whereby the sheath is driven along the body cavity in the extended condition with the rear end region of the sheath drawn along behind the front end region with travel of the instrument along the body cavity.

In another aspect of the present invention there is provided a method of locating an elongated inflatable sheath in position along a body cavity of a patient, wherein the sheath has a closed front end region blocking passage through the sheath and a longitudinal internal passageway that is separate from an interior lumen of the sheath and which extends from an opposite rear end region of the sheath toward the closed front end region, the method comprising:

(a) fitting the sheath on an instrument for driving the sheath along the body cavity in an extended condition in which the front end region of the sheath is forward of the rear end region and such that the instrument is received in the internal passageway;

(b) introducing the instrument into the body cavity; and

(c) feeding the instrument a desired distance into the body cavity whereby the sheath is driven along the body cavity in the extended condition with the rear end region of the sheath drawn along behind the front end region with travel of the instrument along the body cavity.

In still another aspect of the present invention there is provided a method of locating an elongated sheath in position along a body cavity of a patient, wherein the sheath has a front end region adapted for being connected with an instrument for driving the sheath along the body cavity, and an opposite rear end region, and the method comprises:

(a) connecting the front end region of the sheath with the instrument such that the sheath lies alongside the instrument;

(b) introducing the instrument into the body cavity for driving the sheath along the body cavity in an extended condition in which the front end region of the sheath is forward of the rear end region; and

(c) feeding the instrument a desired distance into the body cavity whereby the sheath is driven along the body cavity in the extended condition with the rear end region of the sheath drawn along behind the front end region with travel of the instrument along the body cavity.

Any instrument suitable for driving the sheath along the body cavity may be utilised in a method of the invention. The instrument may be adapted for vibrating or otherwise for being coupled to a vibration source for vibrating the instrument to assist in driving the instrument and hence the sheath along the body cavity.

The instrument may for instance be a push rod or an instrument for examining the interior of the body cavity such as an endoscope. Preferably, the instrument is an endoscope. The endoscope may be selected from the group consisting of a colonoscope, fibroscope, gastroscope, laproscope or other such instrument for viewing the interior of the body cavity.

In methods in which an instrument adapted for viewing the interior of the body cavity is inserted into the sheath for driving the sheath along the body cavity, at least a section of the front end region of the sheath will typically be transparent to enable viewing through the sheath using the instrument.

When the sheath is in the form of an inflatable balloon, being able to view the interior of the body cavity via the instrument facilitates locating of the sheath in the desired position along the body cavity for tamponade a bleed site or effecting examination or other treatment within the body cavity.

Hence, in another aspect of the present invention there is provided a method of locating a flexible elongated sheath in position along the body cavity of a patient utilising an instrument for viewing the interior of the body cavity via a front end region of the instrument, wherein the sheath has a closed front end region blocking passage through the sheath and an opposite rear end region, wherein at least a section of the front end region of the sheath is transparent, and the method comprises:

(a) fitting the sheath on the instrument for driving of the sheath along the body cavity in an extended condition in which the front end region of the sheath is forward of the rear end region, and such that the instrument is received within the sheath;

(b) introducing the instrument into the body cavity, and

(c) feeding the instrument a desired distance into the body cavity whereby the closed end region of the sheath is driven along the body cavity with travel of the instrument and the rear end region is drawn along behind;

wherein the front end region of the instrument is aligned with the transparent said section of the front end region of the sheath for enabling viewing of the interior of the body cavity for determining where to position the sheath along the body cavity.

In another aspect of the invention there is provided a method of locating a flexible elongated sheath in position along the body cavity of a patient utilising an instrument for viewing the interior of the body cavity via a front end of the instrument, wherein the sheath has a closed front end region blocking passage through the sheath and a longitudinal internal passageway that is separate from an interior lumen of the sheath and which extends from an opposite rear end region of the balloon toward the closed front end region thereof, wherein at least a section of the leading end region of the sheath is transparent, and the method comprises:

(a) fitting the sheath on the instrument for driving of the sheath along the body cavity in an extended condition in which the front end region of the sheath is forward of the rear end region, and such that the instrument is received in the internal passageway;

(b) introducing the instrument into the body cavity; and

(c) feeding the instrument a desired distance into the body cavity whereby the closed end region of the sheath is driven along the body cavity with travel of the instrument and the rear end region is drawn along behind;

wherein the front end region of the instrument is aligned with the transparent said section of the front end region of the sheath for enabling viewing of the interior of the body cavity for determining where to position the sheath along the body cavity.

In still another aspect of the present invention there is provided a method of locating a flexible elongated sheath in position along a body cavity of a patient utilising an instrument for viewing the interior of the body cavity, wherein the sheath has a front end region and an opposite rear end region, and the method comprises:

(a) introducing the instrument into the body cavity; and

(b) feeding the instrument a desired distance into the body cavity whereby the sheath is driven along the body cavity by the instrument in an extended condition in which the rear end region of the sheath is drawn along behind the front end region with travel of the instrument along the body cavity;

wherein the instrument is used to view the interior of the body cavity while the sheath is in the body cavity to determine where to position the sheath along the body cavity.

In another aspect of the present invention there is provided an elongated inflatable sheath having a closed front end region for blocking passage through the balloon and an opposite rear end region, and being adapted for being driven into position along a body cavity of a patient in a substantially extended condition in which the closed front end region of the sheath is forward of the opposite end region by an instrument suitable for insertion into the body cavity.

In a yet further aspect of the present invention there is provided a flexible elongated sheath adapted for being driven into position along a body cavity of a patient by an instrument suitable for insertion into the body cavity, wherein the sheath has a closed front end region blocking passage through the sheath and a longitudinal internal passageway for receiving the instrument and extending from the opposite rear end region of the sheath toward the front end region of the sheath.

In still another aspect of the present invention there is provided a flexible elongated sheath for positioning along a body cavity of a patient by an instrument for insertion into the body cavity, wherein the sheath has a front end region and an opposite rear end region, and the front end region of the sheath is adapted for being connected with the instrument such that the sheath lies alongside the instrument for being driven along the body cavity by the instrument in a substantially extended condition in which the front end region of the sheath is forward of the rear end region of the sheath.

In this instance it is not necessary that the front end region of the sheath be closed ended and embodiments in the form of inflatable balloons having a through passageway extending from the rear end region to the front end region for facilitating passage through the balloon thereby ensuring that the balloon is not blocked by the balloon when inflated in position in the body cavity may be provided.

A sheath of the invention may also have a plurality of separate inflatable compartments arranged one in front of another along the sheath and each being able to be selectively inflated.

Preferably, a front said compartment is at least partially inflated to anchor the sheath in the body cavity for facilitating withdrawal of the instrument from the body cavity.

Hence, in a yet further aspect of the present invention there is provided a method of locating a flexible elongated inflatable sheath in position along a body cavity of a patient, wherein the sheath has a front end region and an opposite rear end region, and a plurality of separate inflatable compartments arranged one in front of another along the sheath and each being able to be selectively inflated, and the method comprising:

(a) introducing an instrument into the body cavity for driving the sheath along the body cavity in an extended condition in which the front end region of the sheath is forward of the rear end region;

(b) feeding the instrument a desired distance into the body cavity whereby the sheath is driven along the body cavity in the extended condition with the rear end region of the sheath drawn along behind the front end region with travel of the instrument along the body cavity,

(c) inflating at least one of the inflatable compartments of the sheath to anchor the sheath in position in the body cavity; and

(d) withdrawing the instrument from the body cavity leaving the sheath behind in the body cavity.

In another aspect of the present invention there is provided a flexible elongated sheath for positioning along a body cavity of a patient by an instrument for insertion into the body cavity, wherein the sheath has a front end region and an opposite rear end region, and a plurality of separate inflatable compartments arranged one in front of another along the sheath and each being able to be selectively inflated, and wherein the sheath is adapted for being driven along the body cavity by the instrument in an extended condition in which the front end region of the sheath is forward of the rear end region.

Preferably, a sheath of the invention will be provided with at least one conduit extending along the sheath that provides a passageway from exterior of the rear end region of the sheath to exterior of the front end region of the sheath.

Accordingly, in another aspect of the present invention there is provided a flexible elongated sheath having a front end region and an opposite rear end region, and adapted for being driven into position along a body cavity of a patient in a substantially extended condition in which the front end region of the sheath is forward of the rear end region, and wherein the sheath comprises at least one conduit lying along the sheath that provides a passageway from exterior of the rear end region of the sheath to exterior of the front end region of the sheath.

Preferably, the sheath will have a plurality of such conduits, at least one being a fluid delivery conduit for delivering a physiological wash fluid from an external source in use to the exterior of the front end region of the sheath.

Preferably, the fluid delivery conduit will be arranged for directing at least some of the wash fluid onto the exterior of the front end region of the sheath.

Preferably, the sheath will be provided with at least one additional said conduit for enabling access to forward of the front end region of the sheath. Preferably, an additional conduit for the application of suction to the body cavity will be provided thereby facilitating the removal of liquid from the body cavity to the exterior of the patient. Most preferably, at least one further conduit will also be provided for passage of an implement to the interior of the body cavity forward of the sheath when positioned in the body cavity in use.

The implement may be for measuring physiological parameter of the patient such as temperature or pH, an implement for treating the patient such as for cauterising a wound or bleed site in the body cavity or removing a polyp or other tissue, or an instrument for taking a biopsy from the patient.

Preferably, the sheath will have a number of additional said conduits for receiving different implements, one implement to each additional conduit, respectively.

The sheath may be made of any material deemed suitable for being inserted into the body cavity of the patient. Particularly suitable materials include vinyl polymers, latex, polypropylene including ultra high density polypropylene, polyethylene including linear low polyethylene, polyurethane, neoprene and other plastics material. Preferably, the sheath will be substantially inelastic for inhibiting stretching of the sheath and thereby drag on the sheath as the sheath is driven along within the body cavity.

The methods and sheaths of the invention find application in both medical and veterinary fields and accordingly, the term “patient” is to be taken to include humans and non-human animals such as those of the ape, equine, bovine and ovine families. Typically, the patient will be a human being.

The body cavity of the patient may for instance be that of the uterus, bladder, oesophagus, stomach, nasal cavity, intestine, colon, or gastrointestinal tract of the patient particularly the lower gastrointestinal tract.

Advantageously, an embodiment of a sheath of the invention may be positioned along the body cavity for tamponading the surrounding tissue defining the body cavity or for otherwise treating the patient.

The features and advantages of invention will become further apparent from the following detailed description of preferred embodiments of the present invention and accompanying drawings.

### BRIEF DESCRIPTION OF THE ACCOMPANYING DRAWINGS

FIG. 1 is a schematic view of a sheath received by an instrument for locating the sheath in position along a body cavity in a patient;

FIG. 2 shows the sheath of FIG. 1 when inflated;

FIG. 3 is a schematic view of a front end region of the sheath of FIG. 1 fitted on the instrument;

FIG. 4 is a schematic view illustrating withdrawal of the instrument following positioning of the sheath of FIG. 1 within the body cavity and inflation of the sheath;

FIG. 5 is a schematic view showing another embodiment of a sheath of the invention carried on an instrument for locating the sheath along within a body cavity,

FIG. 6 is a schematic view of yet another embodiment of a sheath of the invention carried on an instrument for locating the sheath in position along within a body cavity of a patient; and

FIG. 7 is a schematic view showing the sheath of FIG. 6 when inflated.

### DETAILED DESCRIPTION OF PREFERRED EMBODIMENTS OF THE INVENTION

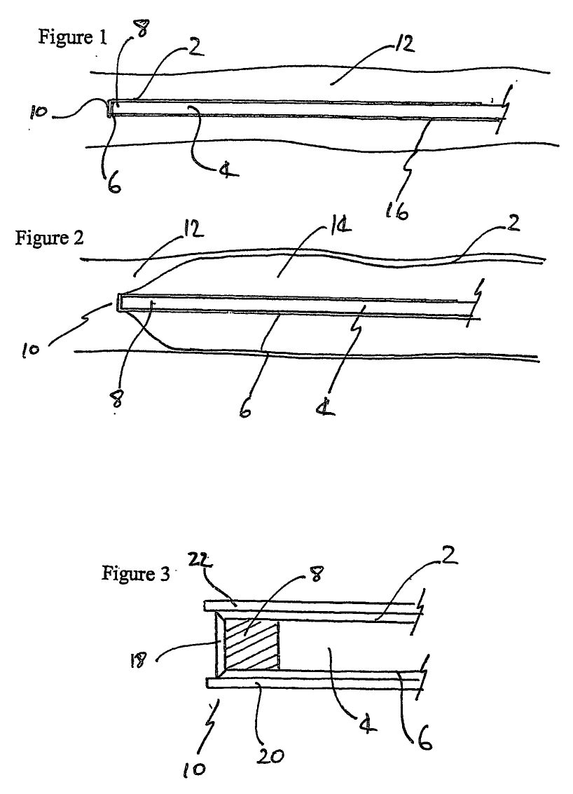

The apparatus shown in FIG. 1 comprises a flexible sheath in the form of an elongate substantially inelastic balloon 2 fitted on an endoscope 4. The endoscope 4 is received in an interior longitudinal passageway 6 of the balloon 2 such that the front end 8 of the endoscope is pressed against the interior of the front end 10 of the balloon. The interior passageway 6 has a diameter larger than that of the external diameter of the endoscope to facilitate insertion and withdrawal of the endoscope from the balloon. Fitting of the balloon on the endoscope 4 is achieved by simply inserting the leading end 8 of the endoscope into the opening to the interior passageway located in the trailing end region of the balloon (not shown) and sliding the endoscope the entire length of the balloon.

Placement of the deflated balloon 2 along within the body cavity, in this instance the lower gastrointestinal tract 12, is achieved by feeding the endoscope along the tract in the conventional manner. As will be appreciated, the balloon 2 is drawn along with the endoscope while being fed along the tract by virtue of the front end 10 of the balloon covering the front end 8 of the endoscope.

Once the endoscope is in position, the sealed lumen 14 of the balloon is inflated via an inflation tube (not shown) provided on the rear region of the balloon and which opens into the lumen, causing the balloon to expand radially and press against the surrounding wall 16 defining the tract 12. By doing so, the balloon acts to tamponade bleed sites in the surrounding wall 16.

At least that portion of the balloon for being inserted into the body cavity will have a substantially constant diameter therealong when the balloon is in a fully inflated condition external of the body cavity. This ensure that a substantially constant pressure is applied to the surrounding wall defining the body cavity along the length of the balloon. In addition, it is not necessary to know the exact location of a bleed site in order to achieve tamponading of the site. Rather, all that is necessary is that the forward end region of the balloon be moved into position past the bleed site or other site to be treated. Typically, at least a major portion of the balloon will have a substantially constant diameter when in a fully inflated condition external of the body cavity.

The balloon will generally be of a length such that some of the inflated portion of the balloon protrudes from the respective body cavity when the balloon is fully inserted into the body cavity. Accordingly, balloons of different lengths and diameters are provided for different applications.

It is not necessary that a balloon be provided with a sealed interior passageway 6, and the endoscope may be inserted directly into the lumen of the balloon. In this instance, the rear end region of the balloon may be clamped to, or otherwise secured around, the endoscope to thereby effectively seal the lumen for enabling inflation of the balloon. The clamping may be achieved by tightening a hose clamp or other suitable device around the instrument. An adhesive tape may also be wrapped around the rear end region of the balloon to thereby seal the balloon around the endoscope. The clamp may then be located over the tape prior to being tightened. Alternatively, the tape may be used alone.

As will be understood, the balloon may be inflated by pumping air or a relatively physiologically inert gas such as carbon dioxide or nitrogen into the lumen through the inflation tube of the balloon. However, a suitable fluid such as physiological saline is not excluded for achieving inflation of the balloon.

In embodiments in which the endoscope is inserted directly into the lumen of the balloon rather than an internal passageway 6, the inflating fluid may be pumped through the endoscope itself into the lumen to cause inflation of the balloon. The inflating fluid may also be withdrawn from the balloon through the endoscope to cause the balloon to deflate. In this instance, the balloon may not be provided with a separate inflation tube.

The front end region 10 of the balloon is illustrated in FIG. 3. As can been seen, the balloon 2 incorporates a transparent window 18 which overlies the leading end 8 of the endoscope allowing an operator to visualise the interior of the gastrointestinal tract through the endoscospe as the endoscope is fed along the tract. To maintain the window 18 relatively clear for facilitating viewing by the operator, a fluid delivery conduit 20 for delivering a physiologically acceptable fluid such as saline from an external source for washing the window 18 and/or wounds or bleed sites is provided on the exterior of the balloon 2. A suction conduit 22 for withdrawing blood and expended wash fluid under suction to a waste reservoir located exterior of the patients body is provided on the opposite side of the exterior of the balloon 2. Desirably, the fluid delivery conduit 20 is arranged for directing at least some of the wash fluid onto the window 18.

As will be understood, the fluid delivery tube 20 is connected to an external pump for pumping the wash fluid along the washer tube as required, while the suction conduit 22 is connected to an external vacuum pump for drawing the waste fluid from the gastrointestinal tract into a collection trap.

Typically, the balloon will be inflated to a pressure of between 40 mmHg to about 80 mmHg to achieve tamponading. The pressure within the balloon is monitored using a suitable pressure gauge arranged for monitoring pressure in the inflation tube, and be regulated by operation of a pressure release valve arranged for releasing pressure from the balloon. A hand pump or any suitable mechanical or electrical pump may be employed for inflating the balloon 2.

While the tamponade balloon is shown in FIG. 3 as only being provided with a single fluid delivery conduit 20 and suction conduit 22, embodiments may be provided with a plurality of fluid delivery and suction conduits. Similarly, conduits for delivering air or other physiologically acceptable gas to the exterior surface of the window 18 for removing fluid thereon to assist visualisation of the interior of the gastrointestinal tract and/or a region of the tract forward of the tamponade balloon may also be provided, as may be one or more additional conduits for facilitating the passage of implements such as instruments along the balloon. Such instruments may include probes for determining the pH or temperature at the front end 10 of the balloon, or for instance, for collecting a biopsy, removing polyps, or cauterising a site as is known in the art.

The fluid delivery and suction conduits and the like may be integrally formed with the balloon or be heat welded or otherwise bonded to the exterior of the balloon. There is no need for such conduits to be secured to the balloon along its entire length. Rather, only leading end regions of the conduits may be connected to the balloon with the trailing end regions of the conduits being free from the balloon. However, embodiments may be provided where the conduit(s) 20 and suction conduit(s) 22 and the like are provided in the lumen of the balloon. In this instance, the tubes will typically sealingly exit the lumen through the rear end region of the balloon

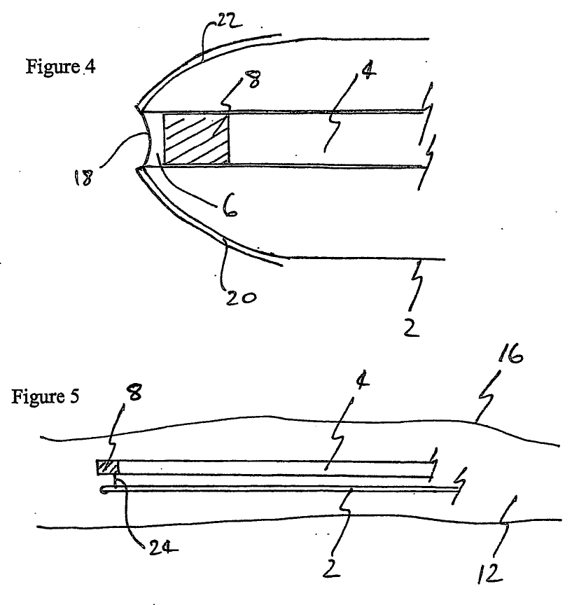

Once the balloon 2 has been located in position and inflated, the endoscope may then be withdrawn from the patient as indicated in FIG. 4 leaving the inflated balloon behind in position lying along the body cavity.

Another embodiment of a tamponade balloon 2 is shown in FIG. 5. In this instance, rather than the balloon receiving the endoscope along its entire length as in the embodiment shown in FIG. 1, this balloon is adapted for lying alongside the endoscope.

To facilitate positioning of the balloon in the gastrointestinal tract by the endoscope, the balloon is provided with a leash 24 of the leading end region 10 of the balloon for securing the balloon to the endoscope. Specifically, the leash is fixedly gripped by an appropriate implement or instrument of the endoscope such that the balloon is caused to be drawn along with the endoscope as the endoscope is pushed along the gastrointestinal tract 12. Once the balloon is in the desired position within the tract, the leash 24 of the balloon is released and the balloon partially inflated to retain the balloon in position while the endoscope is withdrawal, prior to fully inflating the balloon to the desired pressure.

Rather than a leash, the front end region 10 of the balloon may be provided with a hood for reception of the leading end 8 of the endoscope. In this instance, the hood will be of a sufficient length for ensuring retention of the deflated balloon on the endoscope as the endoscope is moved along within the tract during the positioning of the balloon there along. As will be appreciated, the interior dimensions of the hood will be sufficiently large to allow the endoscope to be readily withdrawn from the hood and be removed from the patient leaving the balloon behind in position.

Since the endoscope lies next to the balloon when the balloon is driven along by the endoscope, it is not necessary that the balloon have a closed front end region, and embodiments may be provided having a through passageway extending from the rear end region of the balloon to the front end region of the balloon allowing passage through the balloon when the balloon is inflated in the position in the body cavity. In this instance, the balloon is tubular in form with an annular lumen for reception of the inflating gas.

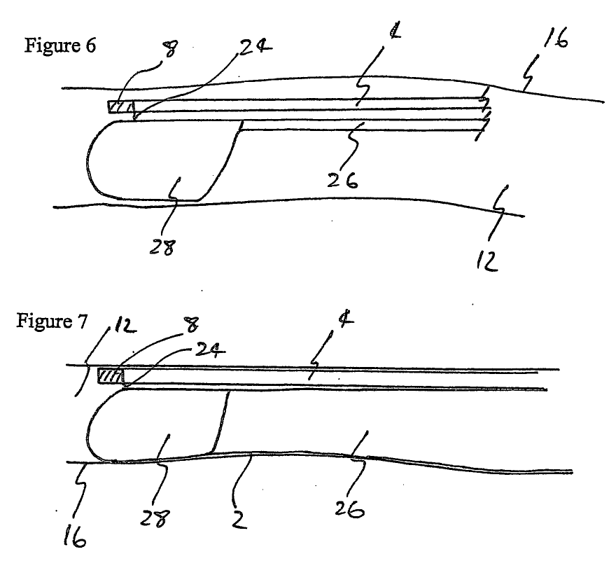

A modified form of the balloon 2 shown in FIG. 5 is illustrated in FIG. 6. In this instance, the balloon is provided with a proximal compartment 26 and a distal compartment 28 which are able to be selectively inflated and deflated, respectively. By inflating the distal compartment 28 of the balloon 2, the balloon may be anchored in the gastrointestinal tract thereby facilitating withdrawal of the endoscope substantially without dislodging the balloon from the desired position. Once the endoscope has been withdrawn, the proximal compartment 26 may be inflated. Embodiments having a through passageway facilitating passage through the balloon when in an inflated condition as described above may also be provided with such separately inflatable compartments.

However, if desired, both the proximal compartment 26 and distal compartment 28 may be at least partially inflated prior to withdrawal of the endoscope 4 as is generally indicated in FIG. 7. By providing a balloon 2 with a number of compartments, pressure may be applied to selected regions of the adjacent wall of the gastrointestinal tract as required, rather than along the entire length of the balloon within the patient's body. As will be understood, each compartment of the balloon will be provided with a corresponding inflation tube for inflation of the compartment, respectively.

Tamponade balloons 2 of the type shown in FIG. 1 with an interior longitudinal passageway 6 for reception of the endoscope may also be provided with a plurality of individual compartments capable of being selectively inflated and deflated. In this instance, air or other suitable gas may be pumped into the interior passageway from the endoscope to facilitate withdrawal of the endoscope from the balloon following partial or complete inflation of one or more of the compartments of the balloon.

Typically, an inflation tube for inflating the balloon or a compartment thereof will also be utilised for escape or withdrawal of the air or gas during deflation of the balloon or compartment thereof.

To assist positioning of a balloon of type shown in FIG. 1 of FIG. 6 in the body cavity and/or to reduce drag, air may be withdrawn from the balloon through the endoscope to cause the balloon to be drawn tightly against the exterior of the endoscope. This inhibits the balloon from sliding along the endoscope should the endoscope need to be drawn backwards along the body cavity during the positioning of the balloon. Holding the balloon against the endoscope in this manner is particularly useful when the balloon is of the type not provided with an interior passageway 6 and the endoscope is inserted directly into the lumen of the balloon.

Indeed, in embodiments provided with a hood which fits over the leading end of the endoscope and the balloon lies next to the endoscope while being driven along the body cavity as described above, this method may be used to effectively secure the hood to the endoscope, prior to pumping air back into the hood from the endoscope to allow the endoscope to be withdrawn.

Withdrawal of the balloon from the patient can be achieved by the medical attendant or physician by drawing the balloon rearwardly from the patient following the deflation of the balloon.

Withdrawal of a balloon of the type shown in FIG. 1 may also be assisted by the provision of one or more cords or the like attached to the leading end of the interior passageway 6 of the balloon and which extend exterior of the patient for being pulled as the pressure within the balloon is reduced gradually such that the extended balloon is progressively peeled from the wall 16 of the gastrointestinal tract 12. That is, the balloon is gradually everted along the interior passageway 6 as it is withdrawn. This significantly reduces friction exerted on the interior surface of the wall 16 and the potential risk of abrasion during the removal of the balloon. This method of withdrawal is particularly preferred when the balloon has been inserted a significant distance into the patient, or extends around a bend or bends when in position within the patient.

To assist positioning and withdrawal of the balloon, the balloon may be coated with a suitable lubricant for minimising abrasion to the surrounding wall of the body cavity. The use of a lubricant also facilitates release of the balloon from the surrounding wall by substantially inhibiting adhesion of the balloon to bleed or wound sites in the wall.

If desired, the exterior of the balloon 2 or regions thereof may be also be coated with an effective amount of one or more suitable substances for inhibiting or preventing further bleeding from the affected site or sites upon being brought into contact with a site as a result of inflation of the balloon. Such substances include coagulants such as thrombin or thrombogenic substances. If appropriate, at least limited regions of the balloon may be coated with a sclerosant or thrombosant agent for causing sclerosis or scarring to inhibit recurrent bleeding.

Similarly, the balloon may be used as a means of delivering therapeutic or other substances in general to the surrounding wall 16 defining the body cavity. Such other substances include radioisotopes, steroids and drugs for treating sites of infection or disease. Diseases which may be treated in this way include cancer. Examples of drugs which may be applied by the balloon include those conventionally used in the treatment of colorectal and other cancers.

Accordingly, the balloon 2 has applications other than as a solely tamponade balloon.

In addition, balloons of the type shown in FIG. 1 may be utilised for assisting internal examination of the patient utilising an endoscope. That is, as the balloon is inflated while the endoscope remains in position, the region of the gastrointestinal tract forward of the endoscope is opened facilitating viewing of the region by the endoscope through the transparent window 18 of the balloon. This may be particularly advantageous for viewing additional regions of, for instance, the sigmoid bend of the lower gastrointestinal tract that may otherwise be inaccessible to the endoscope in the absence of the balloon.

Preferably, a balloon of the invention will be of a sufficient length to extend from the body cavity when located in position. More preferably, the balloon will be adapted for covering that region of the endoscope which remains exterior of the body cavity during the position of the balloon, to thereby protect against contamination of the endoscope. Most preferably, the balloon will be adapted for covering the control section of the endoscope, typically a pistol grip, on which the controls for operating the endoscope are located. In particular, the balloon may be adapted for substantially form fitting over the control section to facilitate access to the controls of the endoscope by the operator. Alternatively, the balloon may incorporate a glove shaped formation for reception of the operators hand and which is arranged for allowing access to and operation of the controls of the endoscope by the operator following insertion of the operator's hand used for operating the controls has been inserted into the glove formation.

The balloon, therefore, may act as a protective sheath which covers the endoscope and protects it from contamination, and so avoids the need for chemical sterilization of the endoscope. As such the balloon has a dual purpose, that is, use as a tamponade or for facilitating examination or other treatment of the patient and secondly, for protecting the endoscope against contamination.

Accordingly, in another aspect of the invention, the balloon may simply receive the endoscope in the manner shown in FIG. 1 including the control section of the endoscope, and the rear end region of the balloon be adapted for being sealed such that the endoscope is enclosed by the balloon and thereby protected against contamination. In this embodiment, the sheath may or may not be adapted for being inflated, and desirably, will be provided for receiving substantially the entire length of the endoscope that is capable of being inserted into the body cavity.

Usually, such a sheath will be provided with one or more conduits as described above such as a fluid delivery conduit 20 and suction conduit 22 for supplying wash fluid and withdrawing waste fluid respectively, and/or a tube or tubes for reception of implements such as a snare for removing polyps or for otherwise examining or treating the patient. The sheath may also be adapted for allowing any conduits or tubes from the endoscope to sealingly pass from the balloon to a pump or vacuum source or the like.

The use of balloons and sheaths as described herein is not restricted to the gastrointestinal tract and embodiments may be provided for use in other body cavities such as in a fallopian tube or for instance the oesophagus. It is also not necessary that an endoscope be used for positioning the balloon and indeed, any suitable instrument may be utilised such as a catheter or for instance a push rod.

Accordingly, although the present invention has been described herein before with reference to a number of preferred embodiments, the skilled addressee will understand that numerous variations and modifications are possible without departing from the scope of the invention.

1. A method of locating a flexible elongated inflatable sheath in position along a body cavity of a patient, wherein the sheath has a closed front end region blocking passage through the sheath in a longitudinal direction thereof and an opposite rear end region, and the method comprises:
(a) introducing an instrument into the body cavity for driving the sheath along the body cavity in an extended condition in which the front end region of the sheath is forward of the rear end region;
(b) feeding the instrument a desired distance into the body cavity whereby the sheath is driven along the body cavity in the extended condition with the rear end region of the sheath drawn along behind the front end region with travel of the instrument along the body cavity; and
(c) withdrawing the instrument from the body cavity leaving the sheath behind in position in the body cavity.

2. A method according to claim 1 wherein the sheath is an inflatable balloon, and the method further comprises inflating the balloon to thereby anchor the balloon in position in the body cavity, prior to removing the instrument.

3. A method according to claim 2 wherein the balloon is inflated such that the balloon expands radially and presses against a surrounding wall defining the body cavity substantially along the entire length of the balloon within the body cavity.

4. A method according to claim 3 wherein the balloon has a proximal inflatable compartment forward of a distal inflatable compartment and wherein the proximal inflatable compartment and the distal inflatable compartment are selectively inflatable relative to one another.

5. A method according to claim 4 wherein the proximal inflatable compartment and the distal inflatable compartment are selectively inflatable and deflatable relative to one another.

6. A method according to claim 4 comprising inflating the proximal inflatable compartment to thereby anchor the balloon in position.

7. A method according to claim 2 wherein at least a major portion of the balloon has a substantially constant diameter along its length when in a fully inflated condition exterior of the body cavity.

8. A method according to claim 2 wherein the balloon tamponades a bleed site in the surrounding wall when the balloon is inflated.

9. A method according to claim 2 wherein the balloon is coated with a substance and the substance is applied to the surrounding wall when the balloon is inflated.

10. A method according to claim 1 wherein the sheath is substantially inelastic.

11. A method according to claim 1 further comprising fitting the sheath on the instrument prior to driving the sheath along the body cavity in the extended condition using the instrument.

12. A method according to claim 1 further comprising inserting the instrument into an interior lumen of the sheath prior to driving the sheath along the body cavity in the extended condition using the instrument.

13. A method according to claim 1 wherein the sheath has a longitudinal internal passageway that is separate from a surrounding interior lumen of the sheath, and the interior lumen is inflatable relative to the internal passageway for thereby anchoring the balloon in position within the body cavity, and wherein the method further comprises inserting the instrument along the internal passageway prior to driving the sheath along the body cavity in the extended condition using the instrument.

14. A method according to claim 1 wherein the front end region of the sheath is adapted for being connected with the instrument, and the method comprises connecting the front end region of the sheath with the instrument such that the balloon thereby lies alongside the instrument, prior to driving the sheath along the body cavity in the extended condition using the instrument while the sheath and the instrument are in a side by side relationship.

15. A method according to claim 14 wherein the sheath is adapted for being coupled with the sheath and the method comprises coupling the sheath and the instrument to thereby connect the sheath and the instrument together.

16. A method according to claim 15 wherein the sheath is adapted for reception of the instrument for thereby coupling the sheath and the instrument together.

17. A method according to claim 13, comprising inflating the interior lumen surrounding the longitudinal passageway to anchor the balloon in position in the body cavity.

18. A method according to claim 1 wherein at least a portion of the front end region of the sheath is transparent for enabling viewing from within the sheath into the body cavity.

19. A method according to claim 1 wherein the sheath has at least one conduit lying along the sheath and opening to exterior of the sheath at the forward end region of the sheath for enabling access to the body cavity or the exterior of the sheath at the forward end region.

20. A method according to claim 19 wherein the sheath is provided with a fluid delivery said conduit for delivery of a fluid to the exterior of the sheath at the forward end region.

21. A method according to claim 19 wherein the sheath is provided with a suction said conduit enabling the application of suction to the interior of the body cavity for withdrawal of liquid from forward of the sheath to exterior of the body cavity.

22. A method according to claim 19 wherein the sheath has at least one implement access said conduit for enabling passage of an implement to forward of the balloon for performing a task in the body cavity.

23. A method according to claim 22 wherein the implement is a surgical implement for performing a surgical task in the body cavity.

24. A method according to claim 1 wherein the instrument is an instrument for enabling viewing into the body cavity from within the balloon

25. A method according to claim 24 wherein the instrument is an endoscope.

26. A method of examining the interior of a body cavity of a patient utilising an instrument for viewing the interior of the body cavity via a front end region of the instrument, wherein the instrument is received within a flexible sheath having a closed front end region blocking passage through the balloon and an opposite rear end region, and at least a section of the front end region of the sheath is transparent, and the method comprises:
a) fitting the sheath on the instrument for driving of the sheath along the body cavity in an extended condition in which the front end region of the sheath is forward of the rear end region, and such that the instrument is received within the sheath;
b) introducing the instrument into the body cavity; and
c) feeding the instrument a desired distance into the body cavity whereby the closed end region of the sheath is driven along the body cavity with travel of the instrument and the rear end region of the sheath is drawn along behind;
wherein the front end region of the instrument is aligned with the transparent section of the front end region of the sheath for enabling viewing of the interior of the body cavity for determining where to position the sheath along the body cavity.

27. A method according to claim 26 wherein the opposite end region of the sheath is adapted for being secured around the instrument, and the method further comprises securing the opposite end region of the sheath around the instrument for thereby protecting the instrument against contamination.

28. A method according to claim 27 wherein the sheath is an inflatable balloon and the method comprises inflating the balloon to thereby anchor the balloon in position in the body cavity.

29. A method according to claim 28 wherein the balloon is inflated such that the balloon expands radially and is pressed against a surrounding wall defining the body cavity substantially along the entire length of the balloon within the body cavity.

30. A method according to claim 28 wherein at least a major portion of the balloon has an essentially constant diameter along its length when in a fully inflated condition exterior of the body cavity.

31. A method according to claim 26 wherein the sheath is substantially inelastic.

32. A method according to claim 26 wherein the sheath receives substantially the entire length of the instrument that is capable of being inserted into the body.

33. A method according to claim 32 wherein the sheath is adapted for receiving a control section of the instrument for controlling operation of the instrument.

34. A method according to claim 26 wherein the instrument is an endoscope.

35. A method according to claim 34 wherein the sheath is adapted for receiving a grip of the endoscope on which controls for operating the endoscope are located.

36. A method according to claim 26 wherein the sheath has at least one conduit lying along the sheath and opening to exterior of the sheath at the front end region, for enabling access to the exterior of the sheath or the body cavity.

37. A method according to claim 36 wherein the sheath is provided with a fluid delivery said conduit for delivery of a fluid to the exterior of the sheath at the forward end region.

38. A method according to claim 36 wherein the sheath is provided with a suction said conduit enabling the application of suction to the interior of the body cavity for withdrawal of liquid from forward of the sheath to exterior of the body cavity.

39. A method according to claim 36 wherein the sheath has at least one implement access said conduit for enabling passage of an implement to forward of the sheath for performing a task in the body cavity.

40. A method according to claim 39 wherein the implement is a surgical implement for performing a surgical task in the body cavity.

41. An elongated flexible sheath for being driven along a body cavity of a patient into position by an instrument, wherein the sheath has a closed front end region and an opposite rear end region for reception of the instrument, and the sheath is adapted for receiving substantially the entire length of the instrument that is capable of being inserted into the body cavity.

42. A sheath according to claim 41 wherein the sheath is an inflatable balloon for being inflated when located in position in the body cavity by the instrument to anchor the balloon in position in the body cavity.

43. A sheath according to claim 42 wherein at least a major portion of the balloon has an essentially constant diameter along its length when the balloon is in a fully inflated condition exterior of the body.

44. A sheath according to claim 41 wherein the sheath is substantially inelastic.

45. A sheath according to claim 41 wherein the rear end region of the sheath is adapted for being secured around the instrument for protecting the instrument from contamination.

46. A sheath according to claim 41 wherein the sheath has a longitudinal interior passageway for reception of the instrument that is separate from a surrounding interior lumen of the sheath.

47. A sheath according to claim 41 wherein the sheath is adapted for receiving a control section of the instrument for controlling operation of the instrument.

48. A sheath according to claim 41 wherein the instrument is an instrument for enabling viewing from within the sheath into the body cavity.

49. A sheath according to claim 48 wherein the instrument is an endoscope and the sheath is adapted for receiving a grip of the endoscope on which controls for operating the endoscope are located.

50. A sheath according to claim 41 wherein the sheath has at least one conduit lying along the sheath and opening to exterior of the sheath at the front end region, for enabling access to the exterior of the sheath or the body cavity.

51. A sheath according to claim 50 wherein the sheath is provided with a fluid delivery said conduit for delivery of a fluid to the exterior of the sheath at the forward end region.

52. A sheath according to claim 50 wherein the sheath is provided with a suction said conduit enabling the application of suction to the interior of the body cavity for withdrawal of liquid from forward of the sheath to exterior of the body cavity.

53. A sheath according to claim 50 wherein the sheath has at least one implement delivery said conduit for enabling passage of an implement to forward of the balloon for performing a task in the body cavity.

54. A sheath according to claim 53 wherein the implement is a surgical implement for performing a surgical task in the body cavity.

55. A sheath according to claim 54 wherein the implement is selected from the group consisting of an implement for cauterising a wound or bleed site, an implement for removing a polyp or for taking a biopsy, and an implement for taking a physical measurement.

56. An elongated balloon for positioning along body cavity of a patient, wherein the balloon has a front end region and an opposite rear end region, and the front end region of the balloon is adapted for being connected to the instrument such that the balloon lies alongside the instrument for being driven along the body cavity by the instrument in a substantially extended condition in which the front end region of the balloon is forward of the rear region of the balloon.

57. A balloon according to claim 56 wherein the balloon is an inflatable balloon for being inflated when located in position by the instrument to thereby anchor the balloon in position in the body cavity.

58. A balloon according to claim 57 wherein at least a major portion of the balloon has a essentially constant diameter along its length when in a fully inflated condition exterior of the body.

59. A balloon according to claim 57 wherein the balloon is substantially inelastic.

60. A balloon according to claim 56 wherein the balloon has at least one conduit lying along the balloon and opening to exterior of the balloon at the front end region, for enabling access to exterior of the balloon or the body cavity.

61. A balloon according to claim 60 wherein the balloon is provided with a fluid delivery said conduit for delivery of a fluid to the exterior of the forward end region of the balloon.

62. A balloon according to claim 56 wherein the balloon is provided with a suction said conduit enabling the application of suction to the interior of the body cavity for withdrawal of liquid from forward of the balloon to the exterior of the body cavity.

63. A balloon according to claim 56 wherein the balloon has at least one implement access said conduit enabling passage of an implement to forward of the balloon for performing a task in the body cavity.

64. A balloon according to claim 63 wherein the implement is a surgical implement for performing a surgical task in the body cavity.

65. An elongated inflatable sheath having a closed front end region and an opposite rear end region, and adapted for being driven into position along a body cavity of a patient in a substantially extended condition in which the front end region of the sheath is forward of the rear end region, and wherein the sheath comprises at least one conduit lying along the sheath and opening to exterior of the sheath at the front end region for providing access to the exterior of the sheath or the body cavity.

66. A sheath according to claim 65 wherein the sheath is an inflatable balloon for being inflated when located in position by the instrument to anchor the balloon in position in the body cavity.

67. A sheath according to claim 66 wherein at least a major portion of the balloon has an essentially constant diameter therealong when the balloon is in a fully inflated condition exterior of the body.

68. A sheath according to claim 65 wherein the balloon is substantially inelastic.

69. A sheath according to claim 65 wherein the sheath is provided with a fluid delivery said conduit for delivery of a fluid to the exterior of the forward end region of the sheath.

70. A sheath according to claim 65 wherein the sheath is provided with a suction said conduit enabling the application of suction to the interior of the body cavity for withdrawal of liquid from forward of the sheath to exterior of the body cavity.

71. A sheath according to claim 65 wherein the sheath has at least one implement access said conduit enabling passage of an implement to forward of the sheath for performing a task in the body cavity.

72. A sheath according to claim 71 wherein the implement is a surgical implement for performing a surgical task in the body cavity.
